(19) United States
(12) Patent Application Publication
(10) Pub. No.: US 2023/0257693 A1
(43) Pub. Date: Aug. 17, 2023

(54) BIOSENSOR

(71) Applicant: ADVANTEST CORPORATION, Tokyo (JP)

(72) Inventor: Kiyoto NAKAMURA, Tokyo (JP)

(21) Appl. No.: 18/304,858

(22) Filed: Apr. 21, 2023

Related U.S. Application Data
(63) Continuation of application No. PCT/JP2021/035911, filed on Sep. 29, 2021.

(30) Foreign Application Priority Data
Oct. 21, 2020 (JP) 2020-176740

(51) Int. Cl. C12M 1/34; G01N 25/48

(52) U.S. Cl. CPC C12M 41/12; G01N 25/48

(57) ABSTRACT

A container holds a liquid sample containing a substrate to be detected. A reactant member is provided in the inner part of the container. The reactant member contains an enzyme and a resin in a state in which they are not mixed.

### CROSS REFERENCES TO RELATED APPLICATIONS

This application is a continuation under 35 U.S.C. § 120 of PCT/JP2021/035911, filed Sep. 29, 2021, which is incorporated herein by reference, and which claimed priority to Japanese Application No. 2020-176740, filed Oct. 21, 2020, the entire content of which is also incorporated herein by reference.

### BACKGROUND

### 1. Technical Field

The present disclosure relates to an evaluation technique for an enzyme activity.

### 2. Description of the Related Art

In order to detect a specific component (substrate) in a liquid sample, a biosensor is employed. As a method for such a biosensor, the colorimetric method has been proposed. A biosensor using the calorimetric method (which will be referred to as a “calorimetric biosensor” or simply as a “calorimetric sensor” hereafter) includes a container or a flow path to hold a liquid sample (which will be simply referred to as the “container” hereafter) and a reactant member provided within the container. The reactant member includes an enzyme that reacts with the substrate contained in the liquid sample. The heat that occurs due to a reaction between the enzyme and the substrate is measured by means of a temperature sensor, so as to identify the kind or the concentration of the substrate.

It is necessary to fix the enzyme in the vicinity of the temperature sensor. Accordingly, with conventional techniques, the enzyme is mixed with a water-soluble light-sensitive resin containing polyvinyl chloride (PVC), and the substrate is coated with the mixed liquid of the enzyme and the resin. Subsequently, the mixed liquid is cured.

As a result of investigating conventional techniques for forming a reactant member by curing a mixed solution of an enzyme and a resin, the present inventor has come to recognize the following problem.

In a case of mixing an enzyme with a resin with a practical concentration for a calorimetric sensor, the mixed solution becomes cloudy. The cloudiness occurs due to agglomeration of the resin components. The reactant member formed by coating with such a cloudy mixed solution leads to non-uniformity of the resin density. Here, examples of non-uniformity include both non-uniformity of the density distribution in a given area and reproducibility (variation) in a case in which many samples are made using the same mixed solution. Uniformity of the density of the reactant member is important for the accuracy and reproducibility of measurement. Thus, improved uniformity of the reactant member is required.

With conventional techniques, an enzyme is immobilized by being captured within a mesh structure of the resin. Accordingly, if the resin has a non-uniform density distribution, the enzyme is strongly immobilized in a portion having high resin density. However, such an arrangement has the potential to involve a problem in that the enzyme dissolves into the liquid if the enzyme is captured in a region having a low resin density. Accordingly, this has the potential to involve a change in the enzyme density contained in the reactant member, leading to degradation in the measurement accuracy and reproducibility.

With the calorimetric sensor, in addition to the heat that occurs due to the reaction between the enzyme and the substrate, heat of wetting that occurs due to the liquid sample infiltrating into the resin is measured as noise. The heat of wetting occurs depending on the ease of infiltration of the liquid sample into the resin, i.e., depending on the resin density. Accordingly, if the resin has a non-uniform resin density for each sample of the reactant member, this leads to variation in the heat of wetting for each reactant member. This leads to degraded reproducibility.

It should be noted that such problems described above are by no means within the scope of common and general knowledge of those skilled in this art. Furthermore, it can be said that the problems described above have been uniquely recognized by the present inventor.

### SUMMARY

The present disclosure has been made in such a situation. Accordingly, it is an exemplary purpose of an embodiment of the present invention to provide a biosensor and a reactor with improved measurement accuracy and/or improved reproducibility.

An embodiment of the present disclosure relates to a biosensor reactor. The biosensor reactor includes: a container structured to hold a liquid sample containing a substrate; and a reactant member provided in an inner part of the container and containing an enzyme and a resin such that they are not mixed.

Another embodiment of the present disclosure relates to a manufacturing method for a biosensor reactor. The manufacturing method includes coating a holding sheet with a resin and curing the resin; and curing the holding sheet coated with an enzyme before the coating with the resin or after the curing of the resin.

It is to be noted that any arbitrary combination or rearrangement of the above-described structural components and so forth is effective as and encompassed by the present embodiments. Moreover, all of the features described in this summary are not necessarily required by embodiments so that the embodiment may also be a sub-combination of these described features. In addition, embodiments may have other features not described above.

### BRIEF DESCRIPTION OF THE DRAWINGS

Embodiments will now be described, by way of example only, with reference to the accompanying drawings which are meant to be exemplary, not limiting, and wherein like elements are numbered alike in several Figures, in which:

FIG. 1 is a cross-sectional diagram showing a schematic structure of a biosensor according to an embodiment;

FIG. 2 is a cross-sectional diagram showing a structure of a reactant member according to an example 1;

FIGS. 3A through 3D are cross-sectional diagrams for explaining a manufacturing method for the reactant member shown in FIG. 2;

FIG. 4 is a cross-sectional diagram showing a structure of a reactant member according to an example 2;

FIGS. 5A through 5D are cross-sectional diagrams for explaining a manufacturing method for the reactant member shown in FIG. 4;

FIG. 6A is a plan view of a container, and FIG. 6B is a cross-sectional diagram showing the container taken along the line I-I;

FIGS. 7A and 7B are cross-sectional views of a reactor according to an example;

FIG. 8A is a waveform diagram showing the output of a temperature sensor in a case in which there is no bubble above the reactant member, and FIG. 8B is a waveform diagram showing the output of a temperature sensor in a case in which there is a bubble above the reactant member; and

FIG. 9 is a cross-sectional view of a reactor according to a modification.

### DETAILED DESCRIPTION

### Outline

An outline of several example embodiments of the disclosure follows. This outline is provided for the convenience of the reader to provide a basic understanding of such embodiments and does not wholly define the breadth of the disclosure. This outline is not an extensive overview of all contemplated embodiments and is intended to neither identify key or critical elements of all embodiments nor to delineate the scope of any or all aspects. Its sole purpose is to present some concepts of one or more embodiments in a simplified form as a prelude to the more detailed description that is presented later. For convenience, the term “one embodiment” may be used herein to refer to a single embodiment or multiple embodiments of the disclosure.

A biosensor reactor according to one embodiment includes: a container structured to hold a liquid sample containing a substrate; and a reactant member provided in an inner part of the container and containing an enzyme and a resin such that they are not mixed.

With this arrangement, the resin and enzyme are not mixed, thereby resolving a non-uniformity due to aggregation of the resin. With such an arrangement employing the biosensor reactor, this provides improved detection accuracy, thereby providing improved reproducibility.

For example, the “state in which an enzyme and a resin are not mixed” means a state in which the enzyme and the resin are separately immobilized. Examples of such a state include: a state in which the enzyme layer and the resin layer are arranged with an interface between them; and a state in which one of the enzymes or the resin is coated with the other.

In one embodiment, the enzyme and the resin may form a layered structure. With such an arrangement in which the first layer film is formed with one of the enzymes and the resin and the first layer is coated with the other, this is capable of preventing the resin and the enzyme from mixing. This provides a uniform resin density.

In one embodiment, the reactant member may include a holding sheet that is any one of fabric, paper, a porous member, and a mesh-structured member. The resin and the enzyme may exist in a state in which they infiltrate into the interior of the holding sheet. This allows the effective surface area of the reactant member to be increased. Such an increase of the surface area leads to an increase of an amount of heat and an increase of heat capacity, thereby facilitating detection by means of a temperature sensor.

In one embodiment, the reactant member may include a holding sheet. Also, the resin and enzyme may be layered on a surface of the holding sheet.

In one embodiment, the reactant member may have a hydrophilicity that is lower than that of a portion surrounding the reactant member. With this, such an arrangement is capable of generating a bubble at a portion that overlaps the reactant member. A bubble has a thermal conductivity that is lower than that of the liquid sample. Accordingly, this is capable of preventing the heat that occurs in the reactant member from escaping. This allows the change in temperature over time accompanying a reaction to be measured.

In one embodiment, the container may be provided with an opening. Also, the opening may be covered by a film. Also, the reactant member may be provided on an inner surface of the film. With such an arrangement in which the container and the film are configured as separable components, this allows the biosensor reactor to be manufactured in a simple manner. Furthermore, this allows the cost to be reduced.

In one embodiment, a surface layer of the film may be coated with a hydrophilic layer. With this, the hydrophilicity of the reactant member becomes relatively lower than that of the surrounding portions. This is capable of generating a bubble.

In one embodiment, the hydrophilicity of the container that faces the reactant member may be lower than that of the surrounding portions. With this, such an arrangement is capable of generating a bubble at a portion that overlaps the reactant member. A bubble has a thermal conductivity that is lower than that of the liquid sample. Accordingly, this is capable of preventing the heat that occurs in the reactant member from escaping. This allows the change in temperature over time accompanying a reaction to be measured.

A biosensor according to one embodiment may include: any one of the biosensor reactors described above; a temperature sensor structured to measure the temperature of the reactant member of the biosensor reactor; and a processing device structured to process an output of the temperature sensor.

A manufacturing method for a biosensor reactor according to one embodiment includes: coating a holding sheet with a resin and curing the resin; and curing the holding sheet coated with an enzyme before the coating with the resin or after the curing of the resin.

Also, the holding sheet may be any one of fabric, paper, a porous member, and a mesh-structured member.

### EMBODIMENTS

Description will be made below regarding preferred embodiments with reference to the drawings. The same or similar components, members, and processes are denoted by the same reference numerals, and redundant description thereof will be omitted as appropriate. The embodiments have been described for exemplary purposes only, and are by no means intended to restrict the present invention. Also, it is not necessarily essential for the present invention that all the features or a combination thereof be provided as described in the embodiments.

In some cases, the sizes (thickness, length, width, and the like) of each component shown in the drawings are expanded or reduced as appropriate for ease of understanding. The size relation between multiple components in the drawings does not necessarily match the actual size relation between them. That is to say, even in a case in which a given member A has a thickness that is larger than that of another member B in the drawings, in some cases, in actuality, the member A has a thickness that is smaller than that of the member B.

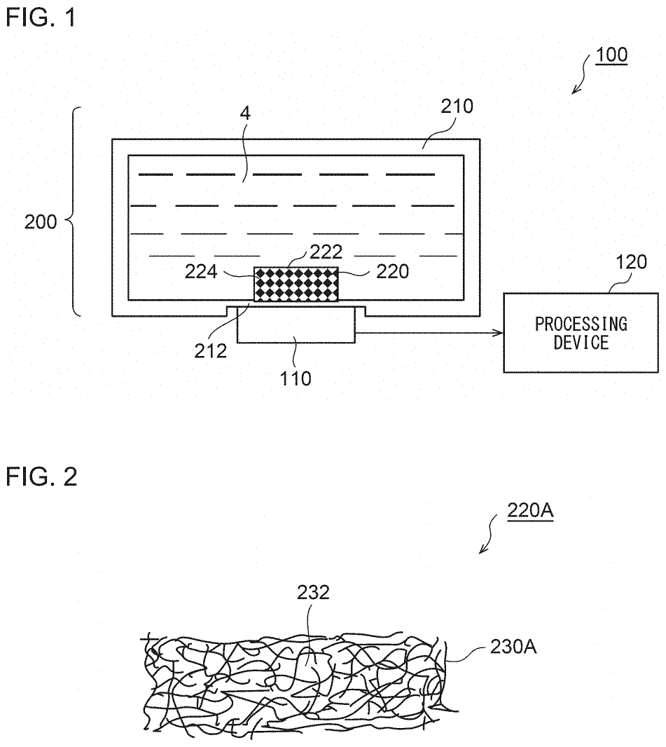

FIG. 1 is a cross-sectional diagram showing a schematic structure of a biosensor 100 according to an embodiment. The biosensor 100 is configured as a sensor employing a calorimetric method. The biosensor 100 measures an amount of a substrate contained in a liquid sample (substrate solution) 4 based on the reaction heat that occurs due to the contact reaction between the substrate and an enzyme (contact catalytic reaction).

The biosensor 100 includes a temperature sensor 110, a processing device 120, and a reactor 200.

The reactor 200 is configured as a test kit of the biosensor 100. The reactor 200 includes a container 210 and a reactant member 220. The container 210 houses a liquid sample (substrate solution) 4 including the substrate.

Specific examples of the liquid sample 4 include blood, urine, sweat, saliva, tears, etc., which are body fluids acquired from a human being. It should be noted that the liquid sample 4 is not restricted to the specific examples described above so long as the liquid sample 4 is a liquid of biological origin. For example, the liquid sample 4 may be a body liquid acquired from other animals, e.g., mammals such as dogs or cats, etc., birds, etc.

Also, the substrate is not restricted in particular. Specific examples of the substrate include glucose, uric acid, lactic acid, proteins, fat, creatinine, bilirubin, etc.

The reactant member 220 is provided in the inner part of the container 210. The reactant member 220 includes an enzyme 222 that can react with the substrate. Specific examples of the enzyme 222 include glucose oxidase, peroxidase, lactate oxidase, trypsin, lipase, creatininase, bilirubin oxidase, etc.

In addition to the enzyme 222, the reactant member 220 includes a resin 224 for holding and immobilizing the enzyme 222 in a stable manner. The material of the resin 224 is not restricted in particular. As the material of the resin 224, a water-soluble light-sensitive resin may be employed, examples of which include polyvinyl chloride (PVC), polyvinyl alcohol (PVOH), and BIOSURFINE (trademark)-AWP manufactured by Toyo Gosei Co., Ltd.

The reactant member 220 holds reaction heat that occurs due to the contact reaction between the enzyme 222 contained in the reactant member 220 and the substrate contained in the liquid sample 4. The temperature sensor 110 measures the temperature of the reactant member 220 of the reactor 200. The structure and the kind of the temperature sensor 110 are not restricted in particular. For example, a thermocouple may be employed.

A portion 212 of the container 210 to be provided with the reactant member 220 has a locally thin structure having a small thickness, which allows the temperature of the reactant member 220 to be measured from the exterior. The temperature sensor 110 is provided on the outer side of the container 210. The temperature sensor 110 measures the temperature of the reactant member 220 via this portion 212. This portion 212 is formed of a material and thickness that do not obstruct heat transfer. With this, there is no need to immerse the temperature sensor 110 in the liquid sample 4. This allows the temperature sensor 110 to be reused.

The processing device 120 processes the output of the temperature sensor 110 so as to estimate the presence or absence of the substrate or an amount of the substrate. As the calculation processing to be employed in the processing device 120, known techniques or techniques that will become available in the future may be employed. Accordingly, detailed description thereof will not be made in the present specification.

In the present embodiment, the reactant member 220 contains the enzyme 222 and the resin 224 in a state in which they do not mix with each other. From another viewpoint, the distribution density of the enzyme 222 in the reactant member 220 is substantially uniform. Furthermore, the density distribution of the resin 224 is also substantially uniform. It should be noted that FIG. 1 shows only a schematic structure of the enzyme 222 and the resin 224. That is to say, the structure and the distribution thereof are not restricted.

The above is the basic structure of the biosensor 100. In the biosensor 100, the reactant member 220 contains the resin 224 with a uniform density distribution as compared with conventional techniques in which a mixed solution of the enzyme 222 and the resin 224 is applied and cured. With such an arrangement in which the resin 224 is distributed with a uniform density distribution, such an arrangement is capable of suppressing variation in the heat of wetting for each sample, thereby providing improved reproducibility. Furthermore, this allows a region having a low density of the resin 224 to be reduced, thereby enabling prevention of dissolution of the enzyme 222. This is capable of suppressing change in the characteristics of the reactant member 220 over time.

Next, description will be made regarding a specific structure and manufacturing method of the reactant member 220.

### Example 1

FIG. 2 is a cross-sectional diagram showing a structure of the reactant member 220A according to an example 1. The reactant member 220A includes a holding sheet 230A having internal cavities 232.

The holding sheet 230A is not restricted in particular. Examples of the holding sheet 230A include non-woven fabric formed of Cupra fiber (Bemcot (trademark) PS-2) manufactured by Asahi Kasei corporation). For example, the holding sheet 230A has a circular structure having a diameter on the order of 1 mm and a thickness on the order of 30 μm. Also, a non-woven wiper such as Wypall manufactured by Nippon Paper Crecia Co., Ltd., may be employed as the holding sheet 230A. However, the present invention is not restricted to such an arrangement. Also, as the holding sheet 230A, non-woven fabrics other than those described above or fabrics other than non-woven fabrics may be employed.

As the holding sheet 230A, paper may be employed instead of such non-woven fabric. Specific examples of such paper that can be employed as the holding sheet 230A include non-woven paper, filter paper, blotting paper, Japanese paper, etc. Also, as the holding sheet 230A, a paper wiper such as Kimwipes or the like manufactured by Nippon Paper Crecia Co., Ltd. may be employed. However, the present invention is not restricted to such an arrangement. With such an arrangement in which the holding sheet 230A is formed of such fabric or paper, this allows the holding sheet 230A to be provided with a low cost.

Also, as the holding sheet 230A, a porous member or a mesh-structured member may be employed instead of such fabric or paper. Specific examples of the porous member that can be employed as the holding sheet 230A include a sponge having a continuous bubble structure. Also, examples of such a mesh-structured member that can be employed as the holding sheet 230A include a mesh member formed of woven thin metal wires having a size on the order of 10 μm.

Also, as the holding sheet 230A, absorbent sheet pieces using capillary action may be employed instead of fabric, paper, porous members, or mesh-structured members.

The resin and the enzyme are held such that they are distributed with a substantially uniform density in a state in which they infiltrate into the internal cavities 232 of the holding sheet 230A. It is difficult to specifically show the structure of the resin and the enzyme held inside the holding sheet 230A. Accordingly, the resin and the enzyme are not shown in FIG. 2.

With such an arrangement employing the reactant member 220A shown in FIG. 2, after the reaction heat that occurs due to the contact reaction between the substrate and the enzyme is stored in the holding sheet 230A, the heat thus stored is slowly diffused. This allows a state in which the reaction heat ΔT can be detected by means of a temperature sensor to be secured for a long period of time. This allows the reaction heat to be detected with good accuracy.

Furthermore, the holding sheet 230A is formed of a material having internal cavities such as non-woven fabric, non-woven paper, a porous member, or the like. A large amount of the enzyme is stored inside the holding sheet 230A. As a result, this increases the contact area between the liquid sample 4 and the enzyme. This is capable of increasing the reaction heat ΔT due to the contact reaction between the substrate contained in the liquid sample 4 and the enzyme.

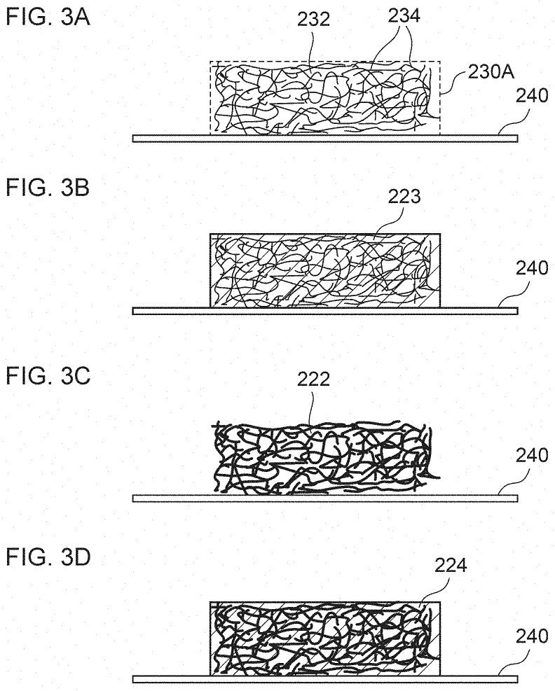

FIGS. 3A through 3D are cross-sectional diagrams for explaining the manufacturing method for the reactant member 220A shown in FIG. 2. In this example, as the holding sheet 230A, non-woven paper or non-woven fabric is employed. The holding sheet 230A has a structure in which fibers 234 are woven. The gaps between the fibers 234 become the internal cavities 232. It should be noted that the shape as viewed in a plan view from above is not restricted in particular. The holding sheet 230A may have a circular shape, rectangular shape, polygonal shape, or any other shape as viewed in a plan view from above. Description will be made assuming that the holding sheet 230A has such a circular shape.

As shown in FIG. 3A, the holding sheet 230A is attached to the upper face of a film 240. The film 240 corresponds to the thin portion 212 of the container 210 shown in FIG. 1. Accordingly, the material and the thickness of the film 240 are designed so as not to obstruct heat transfer. For example, as the material of the film 240, a resin material such as polyester or the like may be selected. The thickness of the film 240 may preferably be determined giving consideration to heat transfer. The film 240 may preferably be configured to have a thickness of 30 μm or less, e.g., on the order of 16 μm.

As shown in FIG. 3B, a liquid 223 obtained by dissolving the enzyme 222 in phosphate buffered solution is dripped onto the holding sheet 230A such that it is infiltrated into its interior using capillary action. Subsequently, the liquid 223 is dried and cured. With this, as shown in FIG. 3C, the enzyme 222 is held in a state of adhesion to the surface of the fibers 234 of the holding sheet 230A.

The film 240 preferably has a hydrophobic surface. This allows the solution dripped onto the holding sheet 230A to be prevented from spreading on the film 240. For example, as the film 240, an adhesive film may be employed. This is capable of immobilizing the holding sheet 230A using adhesion. Furthermore, such an adhesive surface is also hydrophobic, which is convenient.

Next, as shown in FIG. 3D, a solution of the resin 224 such as Biosurfine or the like is dripped onto the holding sheet 230A.

Subsequently, the resin 224 is dried and cured by being irradiated with ultraviolet light.

In the reactant member 220A manufactured using this manufacturing method, the enzyme 222 is adhered to the surface of the fibers of the holding sheet 230A, whereby the enzyme 222 can be provided with a uniform density. Furthermore, the resin 224 uniformly infiltrates into the interior of the holding sheet 230A, whereby the resin 224 can also be provided with a uniform density. Furthermore, with such an arrangement in which, after the enzyme 222 is fixed, the resin 224 is cured, this allows the enzyme 222 and the resin 224 to be held in an unmixed state.

By making microscopic observation directing attention to a given fiber, it can be understood that the enzyme and the resin form a layered structure with the fiber as a base.

### Modification of Example 1

Description has been made in the embodiment regarding an arrangement in which the enzyme 222 is fixed first, and the resin 224 is applied afterward. However, the present invention is not restricted to such an arrangement. Also, the processing order may be swapped. That is to say, first, the resin 224 is dripped onto the holding sheet 230A, infiltrates into the interior using capillary action, and is cured. In this state, the resin 224 is distributed with a uniform density. Subsequently, a solution obtained by dissolving the enzyme 222 in phosphate buffered solution is infiltrated into the holding sheet 230A and is dried. This also provides the enzyme 222 with a uniform density.

### Example 2

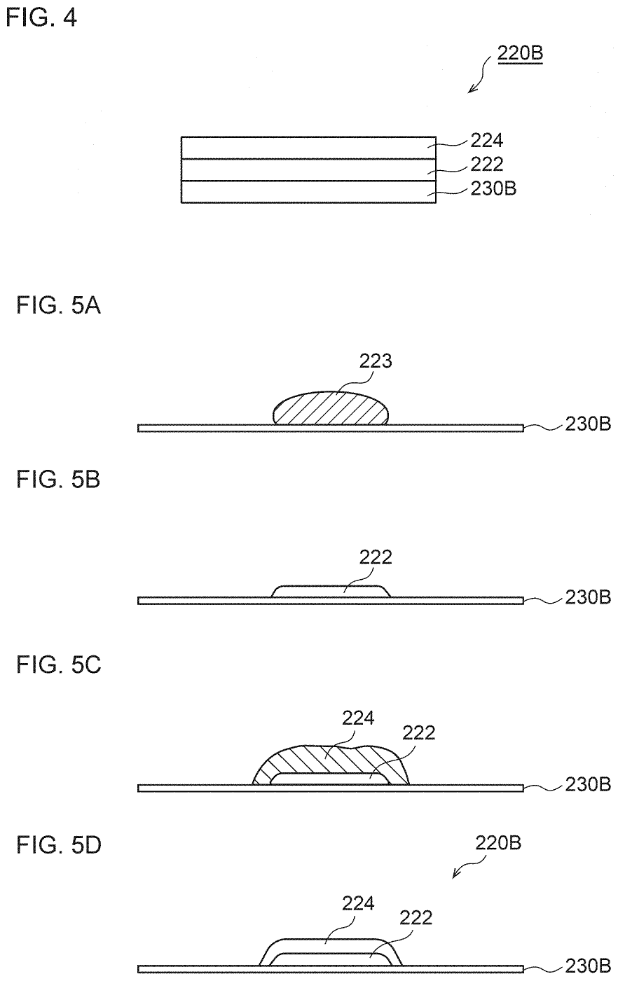

FIG. 4 is a cross-sectional diagram showing a structure of a reactant member 220B according to an example 2. The reactant member 220B forms a layered structure configured of a flat holding sheet 230B, the enzyme 222, and the resin 224.

FIGS. 5A through 5D are cross-sectional diagrams each showing a manufacturing method for the reactant member 220B shown in FIG. 4. In this example, the holding sheet 230B is configured as a film or a tape having a smooth surface having an area that is larger than a region in which the enzyme 222 and the resin 224 are adhered. First, as shown in FIG. 5A, a predetermined region of the sheet 230B is coated with the liquid 223 obtained by dissolving the enzyme 222 in phosphate buffered solution. Subsequently, the holding sheet 230B is dried and cured. With this, as shown in FIG. 3B, a film of the enzyme 222 is formed.

Subsequently, as shown in FIG. 5C, a solution of the resin 224 such as Biosurfine or the like is applied over the enzyme 222 and cured.

With this, as shown in FIG. 5D, the reactant member 220B is completed.

### Example Configuration of Reactor

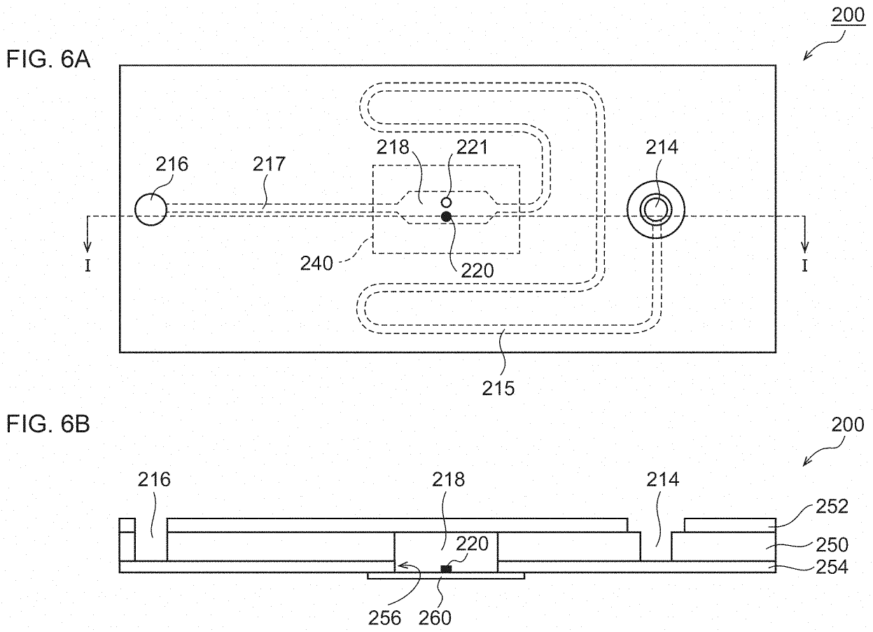

Next, description will be made regarding a specific example of the reactor 200. FIG. 6A is a plan view of the container 210, and FIG. 6B is a cross-sectional view of the container 210 taken along the line I-I. The container 210 has an inlet 214 for the liquid sample 4 and an outlet 216 for the liquid sample 4. An internal space 218 having a large width is formed inside the container 210 so as to hold the liquid sample 4. The internal space 218 and the inlet 214 are formed such that they communicate with each other via a flow path 215. Furthermore, the internal space 218 and the output 216 are formed such that they communicate with each other via a flow path 217.

With such an arrangement in which the flow paths 215 and 217 are formed, this allows the liquid sample 4 to automatically flow into the internal space 218 using capillary action without using a pump or the like. This provides the biosensor 100 with a low cost and a compact size.

As shown in FIG. 6B, the container 210 may have a layered structure. Specifically, the container 210 includes a spacer 250, an upper-side film 252, a lower-side film 254, and a holding film 260.

Openings are formed in the spacer 250 such that they correspond to portions of the internal space 218, the flow paths 215 and 217, the inlet 214, and the outlet 216. Furthermore, openings are formed in the upper-side film 252 such that they correspond to portions of the inlet 214 and the outlet 216. The spacer 250 is arranged such that it is interposed between the upper-side film 252 and the lower-side film 254, so as to form the flow paths 215 and 215 and the internal space 218.

An opening 256 is provided to the lower-side film 254 at a position such that it overlaps the internal space 218. Furthermore, the holding film 260 is attached so as to cover the opening 256. The reactant member 220 is attached to the holding film 260. With this, the reactant member 220 is positioned within the internal space 218.

It should be noted that, in a case in which the reactant member 220 is formed using the manufacturing method shown in FIG. 3, the film 240 shown in FIG. 3 may be employed as the holding film 260. Also, in a case in which the reactant member 220 is formed using the manufacturing method shown in FIG. 5, the holding sheet 230B shown in FIG. 5 may be employed as the holding film 260.

The spacer 250 is configured as a film formed of a resin material such as polyethylene terephthalate (PET) or the like. The spacer 250 has a thickness on the order of 500 μm. However, the present invention is not restricted to such an arrangement.

The upper-side film 252 is configured as a film formed of a resin material such as polyester or the like. The upper-side film 252 has a thickness on the order of 100 μm. However, the present invention is not restricted to such an arrangement. The upper-side film 252 has a lower face (that faces the spacer 250) configured as a hydrophilic face obtained by applying hydrophilic processing to its entire surface. Specific examples of the hydrophilic processing include coating the lower face of the upper-side film 252 with a surfactant or hydrophilic polymer, plasma processing, etc.

The lower-side film 254 is also configured as a film formed of a resin material such as polyester or the like as with the upper-side film 252 described above. The lower-side film 254 has a thickness on the order of 100 μm. However, the present invention is not restricted to such an arrangement. The lower-side film 254 has an upper face (that faces the spacer 250) configured as a hydrophilic face obtained by applying hydrophilic processing to its entire surface. As described above, the film 240 is formed of a resin material such as polyester or the like having a thickness on the order of 16 μm. That is to say, the film 240 has a thickness that is smaller than those of the other films 252 and 254. This allows the temperature of the reactant member 220 to be measured from the exterior with the film 240 interposed.

As shown in FIG. 6A, a dummy reactant member 221 may be provided in the internal space 218 adjacent to the reactant member 220. The dummy reactant member 221 has substantially the same structure as that of the reactant member 220. However, there is a difference between them in that the dummy reactant member 221 contains no enzyme. For example, the dummy reactant member 221 may be manufactured by omitting the processing shown in FIG. 3D. Also, the dummy reactant member 221 may be manufactured by omitting the processing shown in FIGS. 5C and 5D.

The temperature sensor 110 described above is configured to be capable of detecting the difference in temperature between the reactant member 220 and the dummy reactant member 221. The temperature of the reactant member 220 corresponds to the temperature of the liquid sample 4, the reaction heat (rise in temperature) between the enzyme and the substrate, and the heat of wetting. On the other hand, the temperature of the dummy reactant member 221 corresponds to the temperature of the liquid sample 4 and the heat of wetting. Accordingly, by detecting the difference ΔT between the two temperatures, such an arrangement is capable of removing the effects of the temperature of the liquid sample 4 and the heat of wetting. This allows the component of the reaction heat (rise in temperature) to be acquired with high precision.

For example, the temperature sensor 110 may include a first thermocouple that measures the temperature of the reactant member 220 and a second thermocouple that measures the temperature of the dummy reactant member 221. The first thermocouple includes a hot junction (measurement point) that is thermally coupled to the reactant member 220. The second thermocouple includes a hot junction (measurement point) that is thermally coupled to the dummy reactant member 221. In this case, the first thermocouple and the second thermocouple may be configured to have a common cold junction (reference point). In this case, by measuring the electric potential difference between the hot junction of the first thermocouple and the hot junction of the second thermocouple, this arrangement is capable of detecting the temperature difference ΔT.

Next, description will be made regarding further features of the reactor 200.

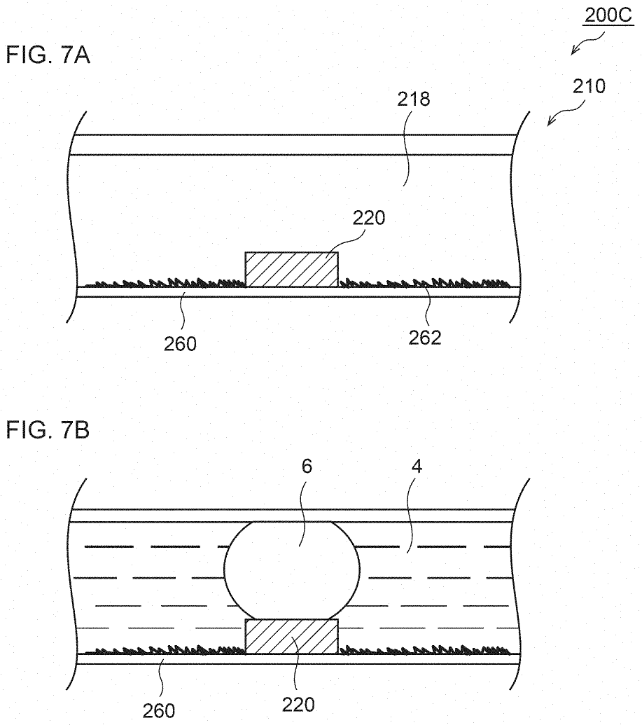

FIGS. 7A and 7B are cross-sectional diagrams each showing a reactor 200C according to one example. In this example, the hydrophilicity of the reactant member 220 is lower than that of portions surrounding the reactant member 220. Specifically, hydrophilic processing is applied to a surface 262 of the holding film 260 for holding the reactant member 220. With this, the hydrophilicity of the surface of the reactant member 220 is relatively low as compared with that of the surrounding portions. Examples of the hydrophilic processing that can be used include coating with a surfactant or hydrophilic polymer, plasma processing, etc.

It should be noted that the entire face that faces the reactant member 220 (upper-side wall face of the internal space) is configured to be hydrophilic. It should be noted that the entire upper face may be configured to be hydrophobic.

FIG. 7B shows a state in which the internal space 218 is filled with the liquid sample 4. The liquid sample 4 readily infiltrates into the region subjected to the hydrophilic processing. In contrast, the liquid sample 4 does not readily infiltrate into the upper space of the reactant member 220. This is capable of generating a bubble 6 on the upper side of the reactant member 220.

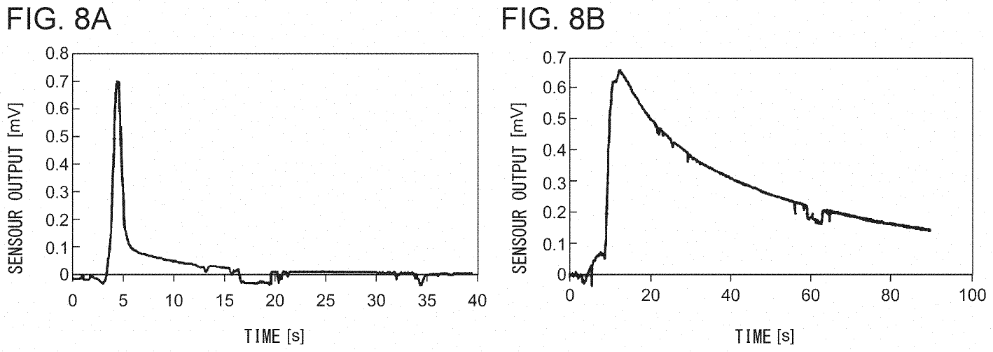

FIG. 8A is a waveform diagram showing the output of the temperature sensor in a case in which there is no bubble above the reactant member 220. FIG. 8B is a waveform diagram showing the output of the temperature sensor in a case in which there is a bubble above the reactant member 220. As shown in FIG. 8A, the temperature of the reactant member 220 rises due to the reaction heat between the enzyme and the substrate. However, in a case in which there is no bubble, the heat diffuses via the liquid sample 4, leading to the temperature falling in a short period of time. In contrast, in a case in which such a bubble occurs above the reactant member 220, this is capable of preventing the heat that occurs in the reactant member 220 from escaping due to the bubble having a thermal conductivity that is lower than that of the liquid sample 4. As a result, as shown in FIG. 8B, this allows the decay time of the output of the temperature sensor to be increased.

As described above, with the reactor 200C shown in FIG. 7, such a bubble is intentionally generated. This is capable of suppressing rapid thermal diffusion. As a result, this arrangement is capable of detecting heat generation information (signal components) that cannot be measured using conventional techniques due to rapid cooling.

Furthermore, the bubble provides an effect of reducing the amount of the liquid that comes in contact with the resin. Accordingly, by controlling the bubble, this arrangement is capable of controlling the reaction amount.

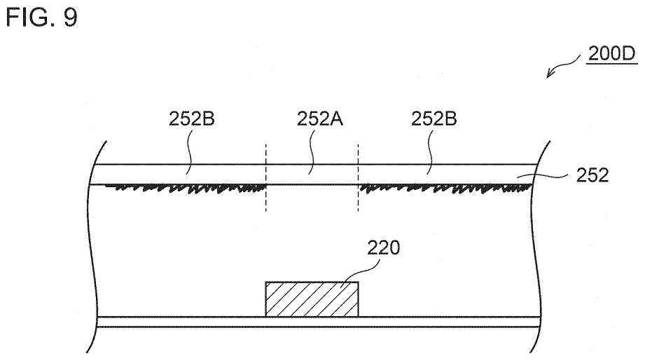

FIG. 9 is a cross-sectional view of a reactor 200D according to a modification. In this modification, the upper-side film 252, which is configured as an upper face of the container 210, has a portion 252A that faces the reactant member 220, and which has a hydrophilicity that is lower than that of a portion 252B surrounding the portion 252A. With this modification, this is also capable of generating a bubble on the upper side of the reactant member 220 when the liquid sample 4 is injected into the internal space 218.

It should be noted that the embodiments have been made for ease of understanding of the present disclosure. That is to say, the description thereof is by no means intended to restrict the scope of the present disclosure. Accordingly, each component disclosed in the embodiments described above is intended to include all changes of design and all equivalents that belong to the technical scope of the present invention.

For example, multiple kinds of enzymes may be immobilized on the same holding sheet. As a specific example in this case, in a case in which glucose is employed as the substrate and glucose oxidase is employed as the enzyme, catalase may be held on the holding sheet in addition to the glucose oxidase. Hydrogen peroxide is generated due to the contact reaction between glucose and glucose oxidase. By further providing a contact reaction between this hydrogen peroxide and catalase, such an arrangement is capable of providing increased reaction heat ΔT.

Also, an arrangement may be made in which the liquid sample contains an enzyme, and a substrate that corresponds to the enzyme is immobilized on the holding sheet. Specific examples of this case include an arrangement in which acid phosphatase is employed as the enzyme, and 1-naphthyl phosphate is employed as the substrate.

Also, a liquid that differs from body fluids may be employed as the liquid sample. Examples of such liquids include liquids obtained from vegetables, fruit, or seaweed.

Description has been made above in the embodiments regarding the biosensor 100 including the temperature sensor 110 and the reactor 200 configured as separable components. However, the structure of the biosensor is not restricted to such an arrangement. The technique according to the present disclosure may be applied to a biosensor including a flow path member and a sensor member integrated as a single unit.

While the preferred embodiments of the present disclosure have been described using specific terms, such description is for illustrative purposes only, and it is to be understood that changes and variations may be made without departing from the spirit or scope of the appended claims.

What is claimed is:

1. A biosensor reactor comprising:
a container structured to hold a liquid sample containing a substrate to be detected; and
a reactant member provided in an inner part of the container and containing an enzyme and a resin such that they are not mixed.

2. A biosensor reactor comprising:
a container structured to hold a liquid sample containing a substrate to be detected; and
a reactant member provided in an inner part of the container and containing an enzyme and a resin such that they each exist with a substantially uniform density distribution.

3. The biosensor reactor according to claim 1, wherein the enzyme and the resin form a layered structure.

4. The biosensor reactor according to claim 1, wherein the reactant member comprises a holding sheet that is any one of a fabric, a paper, a porous member, and a mesh-structured member,
and wherein the resin and the enzyme exist in a state in which they infiltrate into an interior space of the holding sheet.

5. The biosensor reactor according to claim 1, wherein the reactant member comprises a holding sheet,
and wherein the resin and enzyme are layered on a surface of the holding sheet.

6. The biosensor reactor according to claim 1, wherein the reactant member has a hydrophilicity that is lower than that of a portion surrounding the reactant member.

7. The biosensor reactor according to claim 1, wherein the container is provided with an opening,
wherein the opening is covered by a film,
and wherein the reactant member is provided on an inner surface of the film.

8. The biosensor reactor according to claim 7, wherein a surface layer of the film is coated with a hydrophilic layer.

9. The biosensor reactor according to claim 1, wherein a hydrophilicity of the container that faces the reactant member is lower than those of the surrounding portions.

10. A biosensor comprising:
the biosensor reactor according to claim 1;
a temperature sensor structured to measure a temperature of the reactant member of the biosensor reactor; and
a processing device structured to process an output of the temperature sensor.

11. The biosensor reactor according to claim 2, wherein the enzyme and the resin form a layered structure.

12. The biosensor reactor according to claim 2, wherein the reactant member comprises a holding sheet that is any one of a fabric, a paper, a porous member, and a mesh-structured member,
and wherein the resin and the enzyme exist in a state in which they infiltrate into an interior space of the holding sheet.

13. The biosensor reactor according to claim 2, wherein the reactant member comprises a holding sheet,
and wherein the resin and enzyme are layered on a surface of the holding sheet.

14. The biosensor reactor according to claim 2, wherein the reactant member has a hydrophilicity that is lower than that of a portion surrounding the reactant member.

15. The biosensor reactor according to claim 2, wherein the container is provided with an opening,
wherein the opening is covered by a film,
and wherein the reactant member is provided on an inner surface of the film.

16. The biosensor reactor according to claim 15, wherein a surface layer of the film is coated with a hydrophilic layer.

17. The biosensor reactor according to claim 2, wherein a hydrophilicity of the container that faces the reactant member is lower than those of the surrounding portions.

18. A manufacturing method for a biosensor reactor, comprising:
coating a holding sheet with a resin and curing the resin; and
curing the holding sheet coated with an enzyme before the coating with the resin or after the curing of the resin.

19. The manufacturing method according to claim 18, further comprising:
applying hydrophilic processing to a surface of a film; and
attaching the holding sheet to the film.

20. The manufacturing method according to claim 18, wherein the holding sheet is any one of a fabric, a paper, a porous member, and a mesh-structured member.
